(19) United States
(12) Patent Application Publication
(10) Pub. No.: US 2007/0049963 A1
(43) Pub. Date: Mar. 1, 2007

(54) DIRECT VISION PORT SITE DISSECTOR

(76) Inventor: Stephen Kerr, Sunset Beach, CA (US)

Correspondence Address: Pepper Hamilton LLP;One Mellon Center, 50th Floor, 500 Grant Street, Pittsburgh, PA, 15219, US

(21) Appl. No.: 11/551,890

(22) Filed: Oct. 23, 2006

Related U.S. Application Data
(63) Continuation of application No. 10/278,621, filed on Oct. 23, 2002.

(51) Int. Cl. A61M 29/00

(52) U.S. Cl. 606/198

(57) ABSTRACT

A direct vision port dissector for providing safe entry into a body cavity and placement of a standard laparoscopic port for use in laparoscopic surgery. The device comprises an elongate tubular housing within which a laparoscope is placed. Formed at the distal-most opening of the tubular housing is a dissector mechanism that may be selectively deployed and operative to spread tissue in a layer-by-layer fashion while under the direct vision of the laparoscope.

### CROSS REFERENCE TO RELATED APPLICATIONS

This application claims priority to and is a continuation of U.S. Non-Provisional application Ser. No, 10/278,621 filed Oct. 23, 2006, the contents of which are incorporated herein in their entirety.

### BACKGROUND

Laparoscopic surgery is a well-known, widely utilized surgical technique that advantageously reduces patient recovery time due to its minimal tissue damage. Generally, laparoscopic surgery relies upon the formation of one or more puncture wounds through which a body cavity, typically the peritoneal cavity, can be accessed. In this regard, once the peritoneal cavity has been entered, the same is insufflated with carbon dioxide gas, typically to a pressure of approximately 15 mm Hg, followed by the introduction of a laparoscopic port with trocar, which may either be bladed or blunt.

The laparoscopic port is put into the peritoneal. cavity followed by the placement of a laparoscope therethrough to thus provide visualization of the cavity thus enabling the surgeon to view the surrounding organs and conduct the surgical procedure. Advantageously, the use of laparoscopic ports through small diameter openings enables the patient to readily heal following surgery and requires much less recuperation time for the patient as compared to open surgical procedures, which typically deploy long incisions which can and frequently are deemed traumatic to the patient and involve substantially longer recuperative periods.

Despite its advantages, laparoscopic surgery as currently performed can pose substantial risks to the patient. In this respect, it is widely recognized that entry into the peritoneal cavity during laparoscopic surgery, due to the procedure by which the peritoneal cavity is accessed, can cause serious injury to the abdominal organs, such as the spleen, liver and intestine as well as surrounding blood vessels. This risk is due in large part to the fact that in the unoperated abdomen, most surgeons enter the peritoneal cavity using a Veress needle which is pushed blindly through the patient's fascia and peritoneum. The peritoneal cavity is then insufflated followed by the introduction of the laparoscopic port with trocar, which also is pushed blindly into the peritoneal cavity. Once positioned therein, a laparoscope is introduced through the port to thus provide visualization within the cavity.

Problematic with such procedure, however, is the fact that the abdomen is entered blindly on two separate occasions, first through the introduction of the Veress needle and second through the laparoscopic port, which can and on occasion does injure abdominal organs and surrounding blood vessels.

To the extent laparoscopic surgery is performed upon a patient that has previously undergone an abdominal operation, the preferred surgical practice is to enter the peritoneal cavity under direct vision, in this regard, it is known that when a patient has undergone previous abdominal surgery, the abdominal contents can become adherent to the abdominal wall, making blind placement of a Veress needle or trocar too risky of a technique.

According to such technique, the skin is incised and the subcutaneous tissue dissected until the fascia is encountered. The fascia is theft dissected, typically by grasping the fascia with two surgical clamps and incising the fascia sharply followed by successively grasping the subfascial tissue until the peritoneal cavity is entered. Once entered, the laparoscopic port is then placed in the peritoneal cavity under direct vision and the abdomen insufflated with carbon dioxide gas.

Such alternative procedure, however, typically requires a larger skin incision than is typically produced via the use of the Veress needle technique, particularly with respect to obese patients, and is further more prone to gas leakage during surgery, thus requiring constant monitoring and maintenance of adequate insufflation.

In light of such potential complications than can arise via entry into the peritoneal cavity during laparoscopic surgery, attempts have been made to provide means for safely entering into a body cavity utilizing direct visualization, Exemplary of such devices as those disclosed in U.S. Pat. No. 5,441,041, issued to Sauer, et al., entitled Optical Trocar, issued Aug. 15, 1995, which utilizes a blade moveable, between a non-deployed position and a deployed position to thus allow dissection under visualization of an endoscope. Such device, however, does not allow for any type of spreading of the cut tissue to enable the surgeon to see the next layer of tissue to be entered. As such, dissection is performed without prior visualization thereof.

A similar device attempting to provide direct visualization during entry into a body cavity is shown in U.S. Pat. No. 5,569,291, issued to Privitera, et al., entitled Surgical Penetration and Dissection Instrument, issued on Oct. 29, 1996. Such reference discloses a device for forming an entry into a body cavity performed under direct visualization of an endoscope. The dissecting portion of the device consists of a clear plastic conical tip with elevated dissecting blades that is advanced into the tissue via a twisting motion.

The conical tip, however, is advanced bluntly into the tissue before the same can be identified and, as a consequence, incision of the tissue is performed without prior visualization, In fact, inadvertent entry into an organ cannot be avoided via use of such device, and it is only after the organ is entered (and hence damaged, that such matter can be appraised. Moreover, the use of clear plastic has substandard optical visualization due to optical properties inherent in such material, coupled with the conical shape, such that advancement of the tip fails to provide a clear visualization as the same is advanced through tissue.

Other devices that are similar in nature include U.S. Pat. No. 5,720,761, issued to Kalli on Feb. 24, 1998 entitled Visually Directed Trocar and Method; U.S. Pat. No. 5,551,947, issued to Kalli on Sep. 3, 1996, entitled Visually Directed Trocar for Laparoscopic Surgical Procedures and Methods of Using the Same; U.S. Pat. No. 5,609,5621, issued to Kalli on Mar. 11, 1997 entitled Visually Directed Trocar and Method; and U.S. Pat. No. 5,385,572, issued to Nobles, et al. on Jan. 31, 1995 entitled Trocar for Endoscopic Surgery, the teachings of all of which are expressly incorporated herein by reference.

There is thus a substantial need in the art for a system and method that can enable a surgeon to safely enter a body cavity, and in particular the peritoneal cavity, for purposes of performing laparoscopic surgery whereby the surgeon is provided with direct visualization during entry into the cavity such that tissue separation can be visualized and organ and tissue damage can be avoided (i.e., the surgeon can see the tissue prior to dissecting the same). There is additionally need for such a device and system that is capable of forming an entry into a body cavity via a skin incision no greater than that required to admit the introduction of the laparoscopic port and that also preferably forms a tight seal around the port following its introduction such that gas leakage during the laparoscopic surgical procedure is minimized. Still further, there is need for such a system and method which provide for cavity entry without prior insufflation of gas into the cavity but can preferably have a means to insufflate the body cavity following entry, if desired.

### BRIEF SUMMRY OF THE INVENTION

The present invention specifically addresses and alleviates the above-identified deficiencies. In this regard, the present invention is directed to a direct vision port dissector operative to selectively and sequentially dissect through subcutaneous tissue, fascia, pre-peritoneal fat and peritoneum under direct vision of a laparoscope to allow for placement of a laparoscopic port. According to a preferred embodiment, the dissector consists of a long tubular housing having a proximal end and a distal end, the latter being operative to be inserted through a skin incision made upon the patient. Disposed within the distal end is a dissector mechanism having a dissector tip, which is operative to extend from the distal-most opening of the tubular housing and selectively spread apart the various layers of tissue encountered as the distal-most end of the device is advanced through the successive tissue layers and into the peritoneal cavity.

The dissector mechanism is operatively coupled to a handle mechanism extending from the proximal end of the tubular housing which enables the dissecting mechanisms to be selectively controlled, as may be necessary for any anatomical considerations that are visually perceived by the physician. With respect to such handle mechanism, the same preferably takes the form of an actuator bar coupled to a handle member, the latter being operatively coupled to the dissecting mechanism and operative to cause the same to transition between a neutral position, wherein the same is maintained in a coaxial configuration relative said distal end of said housing, and an extended configuration wherein the dissecting mechanism is operative to spread apart at the distal end of the housing and thus spread apart tissue to opposed sides of the distal end of the housing.

According to a preferred embodiment, the dissector tip comprises a pair of arcuate blade members that cooperatively define a generally conical shape. The blades are preferably operatives to extend from the distal-most opening of the housing and extend in diametrically opposed directions to thus produce a spreading motion that extends beyond the opening of the distal end of the tubular housing and thus enables a conventional laparoscope positioned within the housing to provide the physician with direct vision as each layer of tissue is sequentially spread apart from the advancing distal end of the device. To enhance the ability of the laparoscope to view past the dissector tip, the dissector tip may preferably include arcuate voids that define apertures through which the laparoscope can view into the patient when such arcuate blade members assume the general conical shape.

To the extent the distal end of the device comes within close proximity to an organ or other anatomical structure sought to be avoided, the surgeon may take appropriate measures to avoid the same. Otherwise, the physician merely advances the distal end of the device, via the sequential spreading of tissue provided by the dissecting mechanism, until such time as the peritoneal cavity is entered.

Once entered, a conventional laparoscopic port is slid down the shaft of the tubular housing and through the newly dissected incision into the peritoneal cavity, which advantageously can be viewed under direct vision. To enable the device to be utilized with conventional laparoscopic devices and conventional laparoscopic procedure, the same will preferably be made to fit either a ten millimeter port/laparoscope or five millimeter port/laparoscope.

Thereafter, as per conventional laparoscopic procedures, the peritoneal cavity may be insufflated with carbon dioxide gas which may be channeled through the laparoscopic port. In an alternative embodiment of the housing of the present invention, the tubular housing may include a dedicated carbon dioxide channel to thus enable insufflation of the peritoneal cavity to be achieved directly with the dissecting device and prior to the sliding of any laparoscopic port into the peritoneal cavity.

It is therefore an object of the present invention to provide a direct vision port dissector which enables a physician to gain entry into a body cavity, and in particular the peritoneal cavity under direct vision, and thus eliminates the need to blindly enter the same.

Another objet of the present invention is to provide a direct vision port dissector that enables a physician to directly view entry to the peritoneal cavity via a dissection procedure and means of controlling such entry whereby inadvertent dissection of an organ, blood vessel or tissue mass can be avoided or substantially minimized.

Another object of the present invention is to provide a direct vision port dissector which enables a physician to safely gain entry into the peritoneal cavity under direct vision irrespective as to whether or not the patient has or has not undergone previous abdominal surgery.

Another object of the present invention is to provide a direct vision port dissector that can enable a laparoscopic port to be placed into position in a manner that substantially minimizes any possibility of leakage of carbon dioxide once the same in administered to insufflate the body cavity.

Still further objects of the present invention are to provide a direct vision port dissector that, in addition to substantially minimizing the risk of internal organ injury, is of simple construction, easy to use, relatively inexpensive to manufacture, and can be readily deployed utilizing conventional laparoscopic surgical devices and related techniques. The device should also have a means to secure the laparoscope into the device to maintain constant visual orientation and prevent the scope from slipping out of the device during dissection

### BRIEF DESCRIPTION OF THE DRAWINGS

These as well as other features of the present invention will become more apparent upon reference to the drawings wherein:

FIG. 1 is a cross-sectional view of a direct vision port dissector constructed in accordance with the preferred embodiment of the present invention as utilized to gain entry into, the peritoneal cavity of a patient, the port dissector further having included therein a laparoscope to enable entry into the peritoneal cavity to be viewed by a physician.

FIG. 2 is a perspective view taken along line 2-2 of FIG. 1.

FIG. 3 is a cross-sectional view of a laparoscopic port being positioned within the peritoneal cavity of a patient via the direct vision port dissector of the present invention, the latter being withdrawn therefrom.

FIG. 4 is a frontal view of the dissector tip of the direct vision port dissector of the present invention, shown in a first neutral position.

FIG. 5 is a side view of a dissector tip of the direct vision port dissector of the present invention, constructed in accordance with a preferred embodiment, shown in a second operative position.

FIG. 6 is a top view of the dissector tip of FIG. 5 shown in a first neutral position.

FIG. 7 is a frontal view taken along line 7-7 of FIG. 6.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood however, that the same or equivalent functions and sequences may be accomplished by different embodiments and that they are also intended to be encompassed within the scope of the invention.

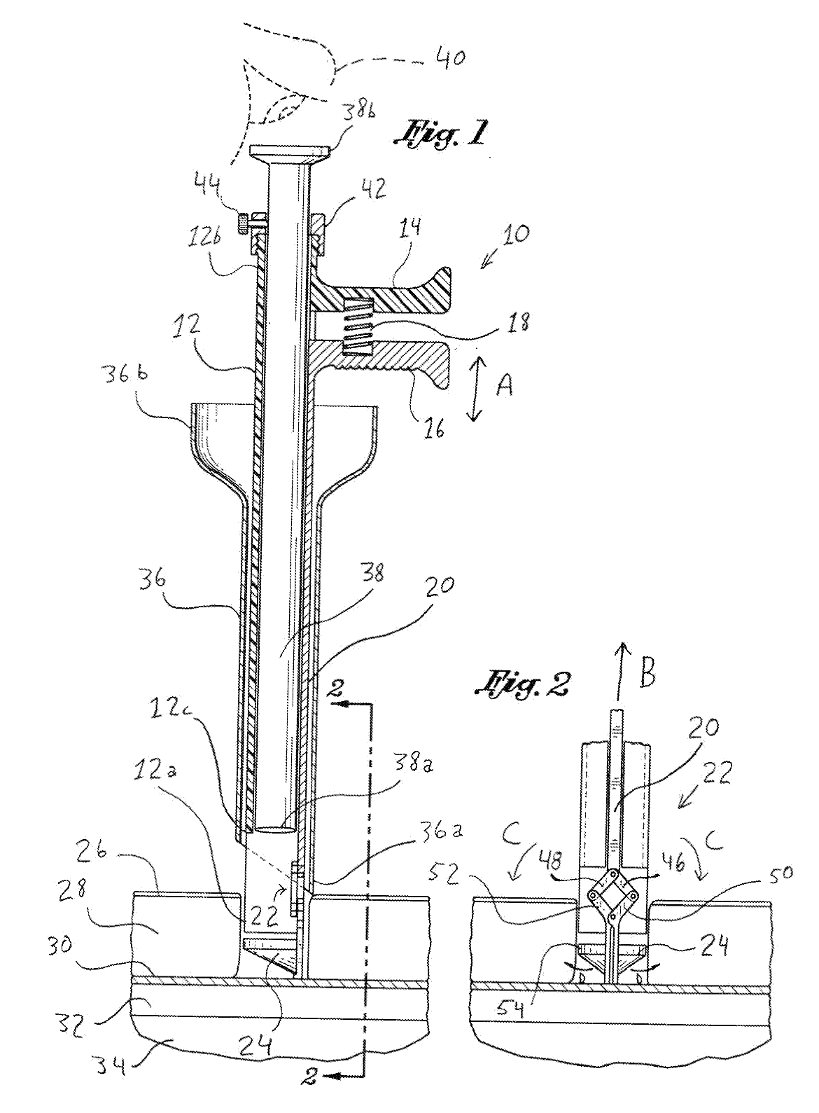

Referring now to the drawings, and initially to FIG. 1, there is shown a cross sectional view of a laparoscopic port site dissector 10 constructed in accordance with a preferred embodiment of the present invention. As illustrated, the device 10 includes an elongate tubular housing 12 having a distal end 12a operative to be inserted within an incision formed upon the skin 26 of a patient and a proximal end 12b for use in coupling with a laparoscope 38 and providing means for manually manipulating the device 10. With respect to the latter, a first handle member 14 is preferably formed upon the distal end, 12b of the housing 12. A second handle member 16 cooperates with handle member 14 to define a handle capable of being selectively compressed in the direction indicated by the letter “A”. Such compressive movement of the handle members 14, 16 facilitates the ability of the device 10 to selectively dissect through tissue, discussed more fully below. In an optional embodiment, a spring member 18 is disposed between handle members 14, 16 to thus cause the handle members 14, 16 to remain in a biased state away from one another.

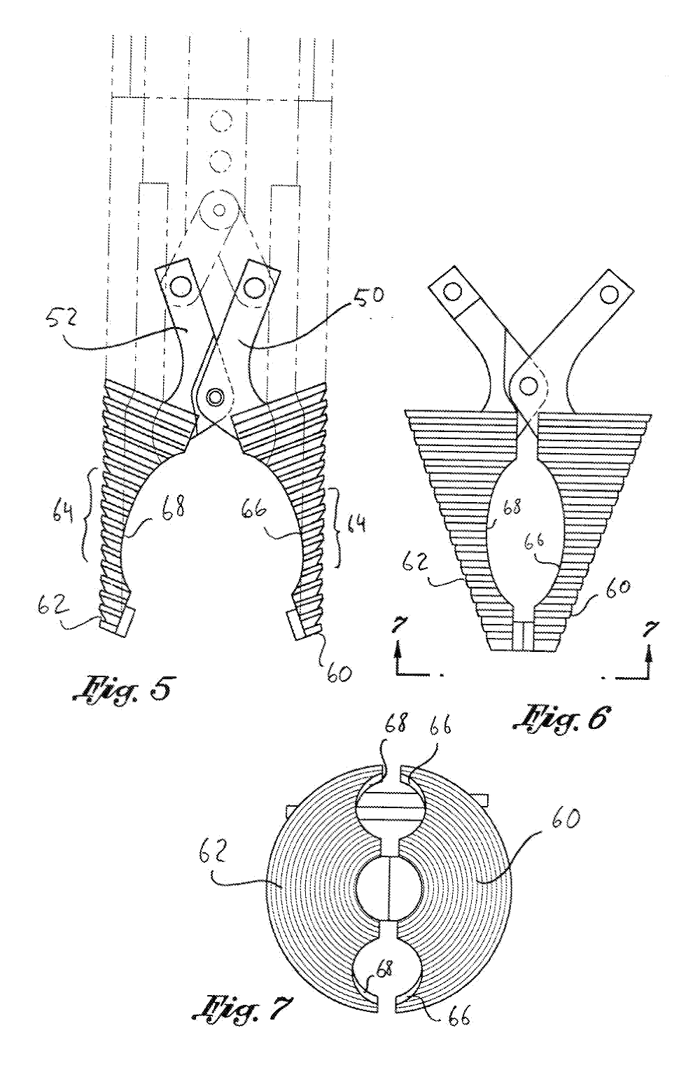

Attached to, or preferably integrally formed with handle 16 is actuator bar 20, the latter extending in general parallel relation to tubular housing 12 and terminating near distal end 12a thereof. Attached to the distal-most end of actuator bar 20 is a dissector mechanism 22 operative to selectively spread tissue, via a dissector tip, the latter preferably comprising an opposed pair of tissue spreaders 24, 54, more clearly seen in FIGS. 2 and 4, to thus enable layers of tissue to be selectively spread apart as the device 10 is advanced through the various layers of tissue, namely, subcutaneous fat layer 28, fascia 30, peritoneum 32 and ultimately into the abdominal cavity 34. Another opposed pair of tissue spreaders 60, 62, which reflect a preferred embodiment of the present invention, are depicted in FIGS. 5-7, discussed more fully below.

Advantageously, the device 10 of the present invention enables such layers of tissue to be selectively penetrated under direct vision by the surgeon to thus enable entry into the peritoneal cavity to be achieved without the risk of damaging organs, vessels, and the like which can and does occur when entry into the abdominal cavity is entered blindly, as per conventional practice.

To achieve that end, the device 10 is operative to receive a laparoscope 38 within the tubular housing 12 thereof to thus provide a physician with the ability to directly view the sequential dissection of the various layers via the dissector 10 of the present invention. As shown in FIG. 1, laparoscope 38 is operatively positioned within the tubular housing 12 such that the distal-most end of the laparoscope 38a is positioned in close proximity to the dissector mechanism 22 and spreader members 54, 25 to thus enable the physician to view each step of the dissection process. To facilitate the ability of the device 10 to receive the laparoscope 38, there is preferably provided a clamp mechanism 42 formed upon distal end 12b of the housing 12, with screw lock 44 to thus enable the same to be locked into position. There may additionally be provided an abutment apparatus or other type of engagement mechanism 12c formed with the lumen of housing 12 to prevent the distal end 38a of the laparoscope from extending beyond distal end 12a of the housing, once secured into position, the eyepiece provided on the proximal end 38b of the laparoscope, as per conventional laparoscopes, enables the surgeon to see and directly view the dissection procedure.

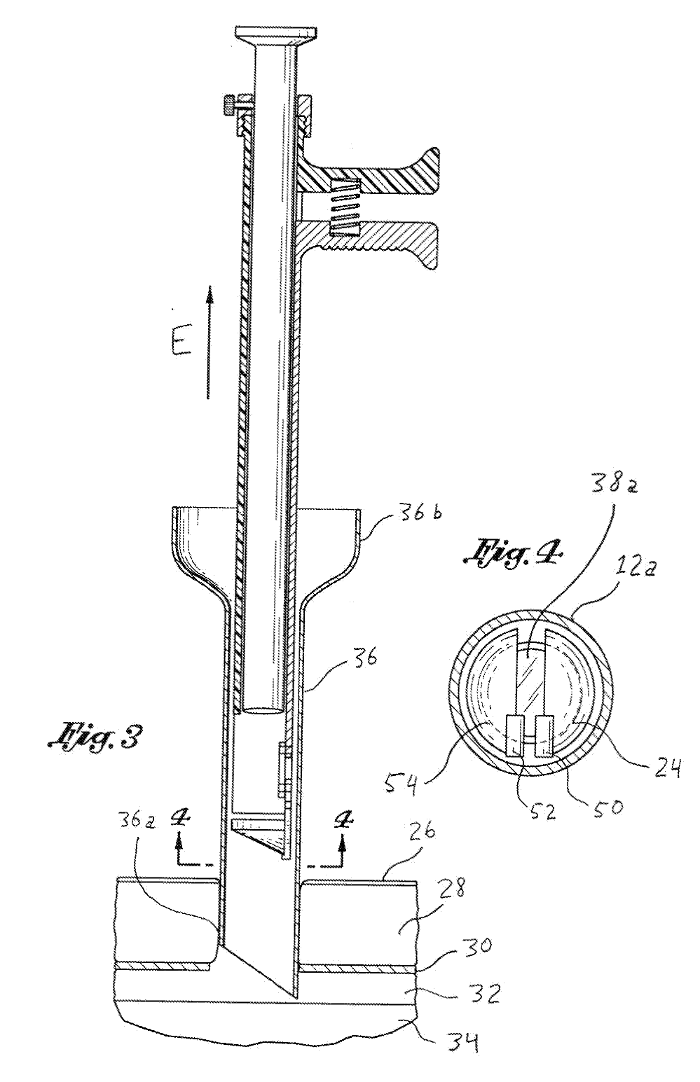

As further shown in FIG. 1, the device 10 is operative to be axially received within the lumen of a conventional laparoscopic port 36 such that once access into the abdominal cavity has been safely achieved, the port 36 may be secured into position so that the subsequent surgical procedure may be performed therethrough, discussed in further reference with respect to FIG. 3.

Referring now to FIGS. 2 and 4, and with initial reference to FIG. 2, there is shown the means by which dissect or mechanism 22 is operative to cause the dissector tip, namely, tissue spreader members 24, 54, to selectively dissect through the various layers of tissue to gain access to the abdominal cavity. As is shown, when actuator bar 20 is retracted in the direction indicated by the letter “B”, which occurs through compression of handle members 14, 16, diagonally extending arm members 46, 48 pivotally connected to the distal end of pull bar 20 are operative to rotate inwardly as indicated by the direction “C”. A second pair of arm members 50, 52, pivotally connected to arms 46, 48, respectively, are then consequently operative to rotate outwardly from the distal-most end 12a of tubular housing 12, as indicated by the direction “D”, to thus cause tissue spreader members 24, 54 to cut through and move tissue away therefrom. In this regard, the tissue spreaders 24, 54, will be operatively transitionable between a neutral position, as shown in FIG. 4, whereby the same are contained within the diameter defined by the distal end 12a of the tubular housing 12 and an operative configuration, whereby the tissue spreader members 24, 54, will extend beyond the circumference defined by the distal end 12a of the tubular housing 12 to thus spread apart the tissue as the distal end 12a of the tubular housing 12 is advanced deeper within the patient.

In use, the device 10 is utilized to sequentially spread layers of tissue 28-32 until such time as the peritoneal cavity is accessed as shown in FIG. 3. To achieve that end, tissue spreaders 24, 54, will preferably comprise arcuate blade members that cooperatively define a generally conical-like structure when the same assume the neutral position, as shown in FIGS. 2 and 4. When the spreader members 24, 54 assume the operative configuration, the same will extend in diametrically opposed directions to thus cause the tissue to be dissected in an outwardly-extending direction relative to the distal end 12a of the housing 12.

Such mode of action is further illustrated in FIGS. 5-7 with respect to alternatively configured tissue spreaders 60, 62, of the dissector tip. As shown in FIG. 5, the tissue spreaders 60, 62 are shown in the operative position whereby the same extend in diametrically opposed directions from the distal end of the tubular housing 12. Such tissue spreaders 60, 62, in contrast to the preferred embodiment depicted in FIGS. 1-4, are provided with serrated edges 64, as may be desired to facilitate the ability of the tissue spreader 60, 62, to advance through tissue.

The tissue spreaders 60, 62, may further be provided with arcuate voids 66, 68, that cooperate to define generally circular or oval-shaped apertures when the tissue spreaders 60, 62, assume the neutral position, as shown in FIG. 6. Advantageously, by providing arcuate voids 66, 68, which define such apertures, there is thus provided channels or access by which the laparoscope 38 (not shown) can directly view the tissue directly ahead of the dissector tip while the tissue spreaders 60, 62 assume a neutral position as shown in FIGS. 6 and 7.

It will be appreciated by those skilled in the art that although depicted as semi-circular blade members, tissue spreaders 24, 54, 60, 62 may take any of a variety of configurations known in the art, and may include any of a variety of tissue spreading mechanisms including additional tissue spreader members. In all cases, however, it is desired that the tissue spreaders be operative to sequentially spread layers of tissue out of the field of vision to be observed by the distal end 38a of the laparoscope 38 to thus enable the physician at all times to see the tissue, during both When the tissue spreader members assume either the neutral or operative configurations until such time as the laparoscopic port 36 is advanced into the channel formed by the dissection of tissue by the tissue spreaders and the device 10 removed therefrom, as shown it FIG. 3.

At all steps during the procedure the distal end 38a of laparoscope 38 is operative to provide the physician with a direct view of the dissection process as the tissue spreader members 24, 54, 60, 62 selectively transition between their neutral position, and the operative tissue spreading configuration. As such, at all times, the physician is able to see each layer of to issue in advance of its dissection and is able to avoid puncturing or otherwise damaging an organ, vessel or other structure. Of further advantage is the fact that the dissector 10 of the present invention is operative to cut and spread apart tissue as the distal end 12a of the housing 12 is advanced axially downward. As a consequence, a snug fit is formed about the tubular housing 12, which in turn provides for a snug fit about the laparoscopic port 36 once the same is ultimately secured into position as shown in FIG. 3.

As will be recognized by those skilled in the art, to the extent dissection can be attained which not only enables the physician to avoid injuring organs, vessels, and the like, but also enables the laparoscopic port 36 to be secured into position with the abdominal cavity in a snug manner to advantageously eliminates or otherwise substantially minimizes any leakage of carbon dioxide gas ultimately used to insufflate the peritoneal cavity. In this respect, not only will entry into the peritoneal cavity be entered in a manner that avoids any risk to any organs, vessels, and the like, it likewise enables a port to be placed into position without the need to provide any sort of insufflation.

Once the laparoscopic port 36 is advanced into the newly dissected incision into the peritoneal cavity, the peritoneal cavity may be insufflated with carbon dioxide as per conventional laparoscopic surgery. The specific laparoscopic procedure may then be performed as per conventional surgical techniques. Along these lines, it is contemplated that the device 10, and note particularly the tubular housing 12, thereof, will be specifically configured to fit with either a ten millimeter port or a five millimeter port it is contemplated, however, that the same may be sized and adapted to fit any of a variety of conventional laparoscopic ports and or adapted to, receive and be utilized with any of a variety of laparoscopes to thus enable the sane to be readily integrated into conventional medical procedures utilizing conventional laparoscopic surgical devices and the like.

Additional modifications and improvements of the present invention may also be apparent to those of ordinary skill in the art. For example, it is contemplated that the device 10 may include a separate port to enable the peritoneal cavity to be insufflated with carbon dioxide, rather than requiring that the device 10 be withdrawn from the laparoscopic port 36 and the carbon dioxide administered separately. Additionally, actuator bar 20 may be configured such that separation of handle members 14, 16 causes such bar 20, via dissector mechanism 22 attached thereto, to selectively dissect through tissue. Thus, the particular combination of parts and steps described and illustrated herein is intended to represent only certain embodiments of the present invention, and is not intended to serve as limitations of alternative devices and methods within the spirit and scope of the invention.

What is claimed is:

1. A laparoscopic direct vision port dissector comprising:
a an elongate tubular housing having proximal and distal ends, said distal end being operative to be inserted within an incision upon a patient, said housing further being operative to receive and secureably hold a laparoscope within the lumen thereof and orient the laparoscope to view through the distal end of said housing;
b. a dissector mechanism formed upon said distal end of said housing, said dissector mechanism having a tissue spreading mechanism formed thereon and operatively transitional between:
(i) a first neutral position wherein said tissue spreading mechanism extends from the distal end of said housing; and
ii) an operative configuration wherein said tissue spreading mechanism cuts across and extends outwardly relative beyond the circumference defined by said distal end of said housing; and
c. a handle mechanism formed upon said proximal end of said housing operative to selectively cause said dissecting mechanism and tissue spreaders to selectively transition between said neutral and operative configurations.

2. The dissector of claim 1 wherein said tissue spreader spreading mechanism comprises opposed blade members operative to extend in diametrically opposed directions from one another at said distal end of said housing when sad dissector mechanism and said spreading mechanism assume said operative configuration.

3. The dissector of claim 2 wherein said dissector further includes a clamp mechanism for securably holding said laparoscope into position within said lumen of said housing.

4. The dissector of claim 3 wherein said clamp mechanism is formed upon said proximal end of said tubular housing.

5. The dissector of claim 1 wherein said tubular housing further includes a stop member formed within the lumen thereof for limiting the distance said laparoscope can extend distally within said tubular housing.

6. The dissector of claim 1 further comprising an actuator bar operatively coupled to said handle mechanism and said dissector mechanism, said actuator bar being operative to cause said dissector mechanism and tissue spreading mechanism to selectively transition between said neutral and operative positions when said handle mechanism is actuated.

7. The dissector of claim 6 wherein said dissector mechanism comprises a first pair of arms pivotally mounted to said pull bar and a second pair of arms coupled to respective ones of said first pair of arms and operative to pivot outwardly relative to said first pair of arms, said second pair of arms having tissue spreader members formed on the respective ends thereof that are operative to transition from said neutral and operative configurations as said first and second arm members pivotally move relative one another.

8. The dissector of claim 1 wherein said dissector further comprises a channel formed therein for administering an insufflative gas.

9. The dissector of claim 1 wherein said dissector is capable of being axially received within a laparoscopic port.

10. The dissector of claim 9 wherein said dissector is insertable through a laparoscope laparoscopic port having a diameter size ranging from approximately ten millimeters to approximately five millimeters.

11. The dissector of claim 2 wherein said opposed blade members cooperate to define a conical-shaped configuration when assuming said first neutral position.

12. The dissector of claim 2 wherein said opposed blade members are provided with serrated cutting edges.

13. The dissector of claim 2 wherein said opposed blade members are provided with at least one void formed thereon defining a at least one channel through which said laparoscope can view through the distal end of said housing.

14. The disssector of claim 13 wherein said at least one void formed upon said opposed blade members cooperate to define said channels at least one channel.

15. The dissector of claim 13 wherein said at least one channel is defined when said tissue spreading mechanism is in said first neutral position.

16. The dissector of claim 1 wherein said dissector mechanism is operative to allow entry though said tissue and into a body cavity under direct vision of the tissue while said tissue spreading mechanism is transitioning between said neutral position and said operative configuration.

17. A laparoscopic direct vision port dissector comprising:
an elongate tubular housing having proximal and distal ends, said distal end being operative to be inserted within an incision upon a patient, said housing further being operative to receive and secureably hold a laparoscope within the lumen thereof and orient the laparoscope to view through the distal end of said housing;
a dissector mechanism formed upon said distal end of said housing, said dissector mechanism having a tissue spreading mechanism formed thereon and operatively transitional between:
(i) a first neutral position wherein said tissue spreading mechanism extends from the distal end of said housing; and
(ii) an operative configuration wherein said tissue spreading mechanism extends outwardly relative to said distal end of said housing; and
a handle mechanism formed upon said proximal end of said housing operative to selectively cause said dissecting mechanism and tissue spreading mechanism to selectively transition between said neutral and operative configurations;
wherein said dissector mechanism is operative to allow entry through said tissue and into a body cavity under direct vision of the tissue.

18. The dissector of claim 17 wherein said dissector mechanism is operative to allow entry through said tissue and into a body cavity under direct vision of the tissue while said tissue spreading mechanism is transitioning between said first neutral position and said operative configuration.

19. The dissector of claim 17 wherein said dissector mechanism is operative to allow entry through said tissue and into a body cavity under direct vision of the tissue while said tissue spreading mechanism is in the neutral position.

20. The dissector of claim 17 wherein said dissector mechanism is operative to allow entry through said tissue and into a body cavity under direct vision of the tissue while said tissue spreading mechanism is in the operative configuration.

21. The dissector of claim 17 wherein said tissue spreading mechanism comprises opposed blade members operative to extend in diametrically opposed directions from one another at said distal end of said housing when said dissector mechanism and said tissue spreading mechanism assume said operative configuration.

22. The dissector of claim 21 wherein said opposed blade members are provided with at least one void formed thereon defining at least one channel through which said laparoscope can view through the distal end of said housing when said tissue spreading mechanism is in said first neutral position.

23. The dissector of claim 22 wherein said at least one void formed upon said opposed blade members cooperate to define said at least one channel.

24. The dissector of claim 17 wherein said tissue spreading mechanism extends beyond the circumference defined by said distal end of said housing while said tissue spreading mechanism is in said operative configuration.

25. A laparoscopic direct vision port dissector comprising:
an elongate tubular housing having proximal and distal ends, said distal end being operative to be inserted within an incision upon a patient, said housing further being operative to receive and hold a laparoscope within the lumen thereof and orient the laparoscope to view through the distal end of said housing;
a dissector mechanism formed upon said distal end of said housing, said dissector mechanism having a tissue spreading mechanism formed thereon, said tissue spreading mechanism operative to perform a controlled dissection of a tissue layer by transitioning between:
(i) a first neutral position wherein said tissue spreading mechanism extends from the distal end of said housing; and
(ii) an operative configuration wherein said tissue spreading mechanism extends outwardly relative to said distal end of said housing and is operative to dissect and spread apart said tissue layer;
said dissector mechanism comprising a handle mechanism, said handle mechanism, comprising an actuator bar operative to selectively transition between said first neutral position and said operative configuration, wherein said laparoscope is capable of viewing said controlled dissection.

26. The dissector of claim 25 wherein said dissector mechanism is operative to allow entry through said tissue and into a body cavity under direct vision of the tissue while said tissue spreading mechanism is transitioning between said first neutral position and said operative configuration.

27. The dissector of claim 25 wherein said dissector mechanism is operative to allow entry through said tissue and into a body cavity under direct vision of the tissue while said tissue spreading mechanism is in the neutral position.

28. The dissector of claim 25 wherein said dissector mechanism is operative to allow entry through said tissue and into a body cavity under direct vision of the tissue while said tissue spreading mechanism is in the operative configuration.
